(19) United States
(12) Patent Application Publication
(10) Pub. No.: US 2007/0053966 A1
(43) Pub. Date: Mar. 8, 2007

(54) Medicated orthopedic support structures for treatment of damaged musculoskeletal tissue

(76) Inventors: Robert Ang, New York, NY (US); Ludwig Weimann, San Diego, CA (US); Anthony Calvert Murrell, Sydney (AU)

Correspondence Address: THORPE NORTH & WESTERN, LLP., 8180 SOUTH 700 EAST, SUITE 200, SANDY, UT, 84070, US

(21) Appl. No.: 11/511,861

(22) Filed: Aug. 28, 2006

Related U.S. Application Data
(63) Continuation-in-part of application No. 10/967,707, filed on Oct. 15, 2004.
(60) Provisional application No. 60/765,253, filed on Feb. 3, 2006.
(60) Provisional application No. 60/512,070, filed on Oct. 17, 2003.

(51) Int. Cl. A61K 9/70; A61K 31/21

(52) U.S. Cl. 424/449; 514/509

(57) ABSTRACT

A system and method for treating damaged musculoskeletal tissue includes an orthopedic support structure and a nitroglycerin-containing composition. The orthopedic support structure provides mechanical support to the damaged tissue and maintains the nitroglycerin-containing composition in contact with the skin of a subject wearing using the system. The nitroglycerin-containing composition can be integrated into the orthopedic support structure or the support structure can be configured to contain removable doses of the nitroglycerin-containing composition. The system and method provides relief of pain and improved function of the musculoskeletal tissue.

The present application claims the benefit of U.S. Provisional Patent Application 60/765,253, file Feb. 3, 2006, and is a continuation-in-part of U.S. patent application Ser. No. 10/967,707, filed Oct. 15, 2004, which claims the benefit of U.S. Provisional Patent Application Ser. No. 60/512,070, filed Oct. 17, 2003, each of which is incorporated herein by reference in its entireties.

### FIELD OF THE INVENTION

The present invention relates to systems and methods for delivering nitroglycerin to damaged musculoskeletal tissue in a body. In particular, the present invention is drawn to systems which provide orthopedic support while transdermally delivering nitroglycerin.

### BACKGROUND OF THE INVENTION

Damaged musculoskeletal tissue is a common and sometimes frequent problem for many active people. The damaged tissue can be a result of overuse, accidental trauma, or other incident. Examples of damaged musculoskeletal tissue include damaged tendons, such as in tendinopathy, broken or bruised bones, pulled or torn muscles, etc. There are a variety of non-operative treatments for each, although none alone has been shown to be universally effective. The non-operative management of many of such injuries involves rehabilitation consisting of relative rest, stretching, and a graduated strengthening exercise program focusing on eccentric tendon loading. Orthopedic support structures, such as braces, splints, sleeves, tapes, wraps, etc, have also been shown to be useful in reducing the force transmitted to the damaged musculoskeletal tissue thereby aiding in the reduction of pain and the overall healing process.

### SUMMARY OF THE INVENTION

The present invention is drawn to a system and method for relieving pain associated with damaged musculoskeletal tissue. The system includes an orthopedic support structure and a nitroglycerin-containing composition. The orthopedic support structure is configured to provide mechanical support to the damaged musculoskeletal tissue and maintains the nitroglycerin-containing composition in contact with the skin. The nitroglycerin-containing composition is formulated to deliver nitroglycerin through a skin surface to the damaged musculoskeletal tissue. The method is related to treating damaged musculoskeletal tissue, and comprises the steps of fitting an orthopedic support structure to a subject which mechanically supports to the damaged musculoskeletal tissue, and applying a nitroglycerin-containing composition to a skin site proximate the damaged musculoskeletal tissue. The composition is capable of delivering nitroglycerin through a skin surface to the damaged musculoskeletal tissue, and the orthopedic support structure is configured to maintain the nitroglycerin-containing composition in contact with the skin.

### DETAILED DESCRIPTION OF THE PREFERRED EMBODIMENT(S)

The present invention provides a system for use in the treatment of damaged musculoskeletal tissue. The system includes the use of an orthopedic support structure and a nitroglycerin-containing composition capable of transdermal delivery upon topical administration. In one embodiment, the nitroglycerin-containing composition is a transdermal patch. The patch is placed in direct contact with a skin surface that is proximate the damaged tissue, and can be replaced periodically over a sufficient period of time to improve force and functional outcome measures at the affected tendon, and/or to relieve pain. In one embodiment the patch is replaced daily (every 24 hours). In another embodiment, a new or replacement patch is placed on a different or new skin site which is also proximate the damaged tissue. In every embodiment, the orthopedic support structure aids in maintaining the nitroglycerin-containing composition in contact with the skin.

The singular forms “a,” “an,” and, “the” include plural referents unless the context clearly dictates otherwise. Thus, for example, reference to a nitroglycerin-containing composition including “an excipient” includes one or more excipients and reference to “the polymer” includes reference to one or more polymers.

As used herein, the terms “formulation” and “composition” are used interchangeably and refer to mixtures, solutions, dispersions, etc., of two or more compounds, fluids, elements, or molecules.

As used herein, the term “musculoskeletal tissue” refers to muscles, bones, ligaments, or tendons in an animal body. As such, the term “damaged musculoskeletal tissue” refers muscles, bones, ligaments, and/or tendons which have been injured or damaged. Non-limiting examples of possible types of damage include a broken or bruised bone; a torn, pulled, or bruised muscle; a tendon with a tendinopathy, etc.

The terms “about” and “approximately” shall generally mean an acceptable degree of error for the quantity measured given the nature or precision of some types of measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, and preferably within 2-fold of a given value. Numerical quantities given herein are approximate unless stated otherwise, meaning that the term “about” or “approximately” can be inferred when not expressly stated.

A “subject,” “patient,” or “mammal” “in need thereof” is an animal that has damaged musculoskeletal tissue. The animal is typically a mammal and more commonly a human.

The terms “treat” or “treatment” means to therapeutically intervene in the development of a disease, disorder, or injury in a subject showing a symptom of this disease, e.g., tendinopathy. In the context of the present invention, these symptoms can include but are not limited to, pain or tenderness, limited range of motion or ability to exert a force on the damaged musculoskeletal tissue without pain, aching of the damaged musculoskeletal tissue at rest, with activities, and/or at night, etc.

The term “improve function” as used herein means significant increases in force outcome measures and/or functional outcome measures. Force and function outcome measures can be determined by any method known in the art. Such measures will vary depending on the type and location of the damaged musculoskeletal tissue.

The term “relieve pain” means improved patient rated pain scores as determined, for example, using the Mann-Whitney rank sum tests. In the context of the present invention, this also refers to subjective determinations such as decreased tenderness of the damaged musculoskeletal tissue, decreased night pain associated with the damaged musculoskeletal tissue, and/or decreased pain with activity.

The phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The terms “continuous” or “continuously” in the context of drug administration refers to a constant, pre-determined amount of drug that is administered over a specified dosing period. A dosing period is the time during which one of the dosage forms in the series is administered to the patient. Accordingly, the dosing regimen will consist of a separate dosing period for administration of each dosage form in the series. Thus, for example, the first dosage form in the series may be worn by the patient for 24 consecutive hours. As one specific example, as used herein, continuous administration refers to delivery of 1.25 mg of nitroglycerin to a subject over 24 hours for successive 24 hour periods for 12-24 weeks. In this context, continuous administration of the preceding transdermal patch requires replacing the patch every 24 hours.

The term “relative release rate,” “flux rate,” or “delivery rate” is determined from the amount of drug released per unit time from e.g., a transdermal delivery system through the skin and into the bloodstream of a subject. Mean relative release rate may be expressed, e.g., as μg drug/hr or, for comparing delivery systems covering skin areas of different size, as μg drug/cm²/hr. For example, a transdermal delivery system that releases 1.25 mg of nitroglycerine over a time period of 24 hours is considered to have an average release rate of about 52.1 μg/hr. For purposes of the invention, it is understood that relative release rates may change between any particular time points within a particular dosing interval, and the term therefore only reflects the overall release rate during the particular dosing interval.

The terms “orthopedic support” or “orthopedic support structure” refers to devices which provide mechanical support to damaged musculoskeletal tissue. The orthopedic support structures can be configured to provide support to any region of the human body including but not limited to fingers, wrists, elbows, arms, shoulders, backs, knees, ankles, legs, and necks. In one embodiment, the orthopedic supports are configured to provide mechanical support to joints. In another embodiment, the orthopedic supports are configured to provide mechanical support to damaged bones. The degree of support provided by the orthopedic support structure can vary from low to moderate support, such as a flexible sleeve or brace, up to and including rigid support, such as traditional fixed orthopedic casts. Generally, any form of orthopedic support structures known in the art can be used so long as it can be specifically configured to maintain the nitroglycerin-containing composition in contact with the skin. It is emphasized that a bandage-like structure, such as a transdermal patch with a backing, is not considered to be an orthopedic support structure in accordance with embodiments of the present invention. Orthopedic support structures must be configured to provide support to damaged musculoskeletal tissue that would be therapeutically useful and supportive of the tissue, even in the absence of the nitroglycerin-containing composition.

As used herein, the term “drug delivery zone” refers to the area of skin which comes into direct contact with the nitroglycerin-containing composition. For example, when the nitroglycerin-containing composition is in the form of a transdermal matrix patch, the drug delivery zone would be the area in which the drug containing matrix contacts the skin.

As extended nitroglycerin use may cause undesirable tolerance issues, in some cases, it can be desirable allow for drug holidays. “Drug holiday(s)” refer to periods of time in which the transdermal patch is removed for a predetermined length of time before a subsequent patch is administered. For example, a nitroglycerin-containing composition of the present invention may be applied to the skin of a subject for a period of 12 hours after which the composition is removed and a drug holiday period of 12 hours is allowed to pass before a subsequent reapplication of more nitroglycerin-containing composition to the subject's skin. Other periods of time for drug delivery and drug holidays can also be implemented, as would be known to those skilled in the art after considering the present disclosure. During the drug holiday periods the use of the orthopedic support can be continued.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity, and thus, should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

Before particular embodiments of the present invention are disclosed and described, it is to be understood that this invention is not limited to the particular process and materials disclosed herein and as such may vary to some degree. It is also to be understood that the terminology used herein is used for the purpose of describing particular embodiments only and is not intended to be limiting, as the scope of the present invention will be defined only by the appended claims and equivalents thereof.

The present invention relates to a system for relieving pain associated with and improving function of damaged musculoskeletal tissue. The system includes an orthopedic support structure which is capable of providing mechanical support to the damaged musculoskeletal tissue and a nitroglycerin-containing composition capable of delivering nitroglycerin through a skin surface to the damaged musculoskeletal tissue. The orthopedic support structure used in the system is configured to maintain the nitroglycerin-containing composition in contact with the skin.

The method is related to treating damaged musculoskeletal tissue, and comprises the steps of fitting an orthopedic support structure to a subject which mechanically supports to the damaged musculoskeletal tissue, and applying a nitroglycerin-containing composition to a skin site proximate the damaged musculoskeletal tissue. The composition is capable of delivering nitroglycerin through a skin surface to the damaged musculoskeletal tissue, and the orthopedic support structure is configured to maintain the nitroglycerin-containing composition in contact with the skin.

The nitroglycerin-containing compositions of the present invention can come in a variety of forms so long as they can provide transdermal delivery of nitroglycerin, i.e. a transdermal dosage form. Transdermal dosage forms are convenient dosage forms for delivering nitroglycerin. Transdermal dosage forms are particularly useful for timed release or sustained release of nitroglycerin. Non-limited examples of transdermal dosage forms which can be used in the present invention include topical solutions, suspensions, ointments, pastes, creams, lotions, gels, patches, and the like. Preparations of such dosage forms are well known in the art and can be formulated using numerous known excipients.

A common transdermal dosage form is a diffusion driven transdermal system (transdermal patch) using either a fluid reservoir or a drug in adhesive matrix system. In one embodiment, the transdermal dosage form is a transdermal patch. In another embodiment, the transdermal patch is a matrix patch. In yet another embodiment, the transdermal dosage form is a gel.

Transdermal dosage forms used in accordance with the invention can, but does not have to, include a backing layer made of a pharmaceutically acceptable material which is generally, but not required to be, impermeable to the nitroglycerin. The backing layer preferably serves as a protective cover for the nitroglycerin, and may also provide a support function for the nitroglycerin-containing composition. In one embodiment of the present invention layer can be integrated into the orthopedic support of the pain relieving system. In another embodiment, the backing layer can include a means for securing the transdermal patch to the orthopedic support structure. Examples of such securing means include but are not limited to adhesives, tapes, heat fusing, or mechanical attaching mechanisms, such as Velcro, etc. In one embodiment, the orthopedic support structure includes a pouch capable of retaining the nitroglycerin-containing composition and maintaining the composition in contact the skin. In another embodiment, the pouch can be closable. In another embodiment, the orthopedic support structure can include a recessed area on the area of the support which would be proximate the skin. The recessed area can be configured to retain the nitroglycerin-containing composition, e.g., patch, gel, or other composition containing nitroglycerin. The nitroglycerin-containing composition could be retained in the recessed area using Velcro, adhesive, sewing means, heat fusing means, tape, or other retaining means.

The backing layers of the patches can be made from any material known in the art so long as it does not interfere with the ability of the nitroglycerin to be delivered through the skin to the damaged musculoskeletal tissue. Examples of suitable materials include but are not limited to films of high and low density polyethylene, polypropylene, polyvinylchloride, polyurethane, polyesters such as poly(ethylene phthalate), metal foils, metal foil laminates of such suitable polymer films, textile fabrics, including fabrics which are impermeable to the nitroglycerin due to their physical properties, and the like. The type of material used in the system of the present invention can vary depending on the nature and physical location of the damaged musculoskeletal tissue. For example, when the orthopedic support is configured to be flexible for application to a joint the backing layer will also be made of a flexible material. When the orthopedic support is configured for application to static musculoskeletal tissue, such as a cast limb, the backing layer can be more rigid.

The backing layer can be any appropriate thickness to provide the desired protection and support functions. A suitable thickness can be from about 10 to about 200 microns. In some embodiments, the orthopedic support structure can act as a backing layer. In such embodiments, the thickness of the backing layer can be the thickness of the orthopedic support structure. Desirable materials and thickness will be apparent to the skilled artisan.

In certain preferred embodiments, the nitroglycerin-containing composition used in accordance with the invention contains a pharmacologically or biologically acceptable polymer matrix layer. Generally, the polymers used to form the polymer matrix are those capable of forming thin walls or coatings through which pharmaceuticals can pass at a controlled rate. A non-limiting list of exemplary materials for inclusion in the polymer matrix includes polyethylene, polypropylene, ethylene/propylene copolymers, ethylene/ethylacrylate copolymers, ethylenevinyl acetate copolymers, silicones, rubber, rubber-like synthetic homo-, co- or block polymers, polyacrylic esters and the copolymers thereof, polyurethanes, polyisobutylene, chlorinated polyethylene, polyvinylchloride, vinyl chloride-vinyl acetate copolymer, polymethacrylate polymer (hydrogel), polyvinylidene chloride, poly(ethylene terephthalate), ethylene-vinyl alcohol copolymer, ethylene-vinyloxyethanol copolymer, silicones including silicone copolymers such as polysiloxane-polymethacrylate copolymers, cellulose polymers (e.g., ethyl cellulose, and cellulose esters), polycarbonates, polytetrafluoroethylene and mixtures thereof. Exemplary materials for inclusion in the polymer matrix layer are silicone elastomers of the general polydimethylsiloxane structures, (e.g., silicone polymers).

Other preferred materials for inclusion in the polymer matrix layer include silicone polymers that are cross-linkable copolymers having dimethyl and/or dimethylvinyl siloxane units that can be crosslinked using a suitable peroxide catalyst. Also preferred are those polymers consisting of block copolymers based on styrene and 1,3-dienes (particularly linear styrene-isoprene-block copolymers of styrene-butadiene-block copolymers), polyisobutylenes, polymers based on acrylate and/or methacrylate.

The polymer matrix layer may optionally include a pharmaceutically acceptable crosslinking agent. A non-limiting example of suitable crosslinking agent is tetrapropoxy silane. The polymer matrix layer can be integrated with the backing layer or the orthopedic support structure.

An adhesive may also be included in the nitroglycerin-containing composition. Examples of adhesives which can be used include but are not limited to hypoallergenic adhesives, such as polyacrylic adhesive polymers, acrylate copolymers (e.g., polyacrylate) and polyisobutylene adhesive polymers. In other preferred embodiments of the invention, the adhesive can be a hypoallergenic and pressure-sensitive contact adhesive.

The nitroglycerin-containing composition of the present invention may also optionally include a permeation enhancing agent. Permeation enhancing agents are compounds that promote penetration and/or absorption of the nitroglycerin through the skin or mucosa and into the blood stream of the patient. A non-limiting list of permeation enhancing agents includes polyethylene glycols, surfactants, and the like. Permeation enhancement can also be achieved by the occlusion of the containing composition. The orthopedic support structures of the present invention can provide the required occlusion.

The nitroglycerin-containing composition may also include a variety of other excipients, including but not limited to, softeners such as higher alcohols such dodecanol, undecanol, and octanol; esters of carboxylic acids, wherein the alcohol component may also be a polyethoxylated alcohol; diesters of dicarboxylic acids, such as di-n-butyladiapate; triglycerides, particularly medium-chain triglycerides of caprylic/caproic acids or coconut oil such as glycerol and 1,2-propanediol; as well as softeners such as levulinic acid and caprylic acid, which can also be esterified by polyethylene glycols. Other excipients may also be included as necessary. Addition of such excipients is within the knowledge of one of ordinary skill in the art.

The dosage of the nitroglycerin according to the present invention can be determined on an individual, case-by-case basis by one of ordinary skill in the art, but generally does not exceed an average delivery rate of nitroglycerin of about 85 mcg/hr. In one embodiment, the nitroglycerin-containing composition is formulated and configured to deliver from about 5 mcg/hr to about 85 mcg/hr of nitroglycerin. In another embodiment, the nitroglycerin-containing composition is formulated and configured to deliver from about 15 mcg/hr to about 75 mcg/hr of an nitroglycerin. In a preferred embodiment, the nitroglycerin-containing composition is formulated and configured to deliver from about 30 mcg/hr to about 65 mcg/hr of nitroglycerin. It is noted that the dosage levels used in the present invention are generally lower than dosage values of nitroglycerin used in transdermally treating angina.

In one embodiment of the present invention, the nitroglycerin-containing composition can be formulated to provide a reduced delivery rate per cm² of the drug delivery zone. The reduced delivery rate per cm² of the drug delivery zone allows for the nitroglycerin-containing composition to contact a relatively large drug delivery zone while delivering relatively small amounts of nitroglycerin.

This being stated, in one embodiment, the nitroglycerin-containing composition can be formulated to provide a delivery rate of nitroglycerin to the drug delivery zone in amounts of from about 1 μg/cm²/day to about 600 μg/cm²/day. In another embodiment, the nitroglycerin-containing composition can formulated to deliver from about 1 μg/cm²/day to about 280 μg/cm²/day. In yet another embodiment the nitroglycerin-containing composition can formulated to deliver from 10 μg/cm2/day to about 280 μg/cm²/day. In a further embodiment the nitroglycerin-containing composition can formulated to deliver from about 50 μg/cm²/day to about 250 μg/cm²/day. The lower delivery rates of the nitroglycerin allow for the increase in the drug delivery zone without increasing the dosage amount delivered to the patient. The lower dosage amounts can also decrease or eliminate some of the side effects which are affiliated with high dosages of nitroglycerin, namely headache, lightheadedness, and hypotension.

As described above, each system of the present invention has an area, known as the drug delivery zone, which is defined to be the area where the nitroglycerin-containing composition contacts an intact human skin surface. The area of the drug delivery zone can vary depending on the desired rate of delivery per cm² of the drug delivery zone and the total dosage amount to be delivered in a given dosing period. The size of the drug delivery zone can be from about 2.5 cm² to about 100 cm². In another embodiment, the size of the drug delivery zone can be from about 3 cm² to about 50 cm².

The orthopedic support structures which can be used in the present invention include braces, compression bandages, structurally supporting tape wraps, elastic sleeves, ceramic, plaster and fiberglass casts, etc. Standard transdermal patches and non-supportive bandages are not included as orthopedic support structures. In one embodiment, the orthopedic support is a brace. Braces are well known orthopedic support structures and varying designs exist for the different regions of the body. Non-limiting examples of brace configurations which can be used in the present invention include knee braces, elbow braces, ankle braces, and wrist braces. When the orthopedic support structure is a brace, the nitroglycerin-containing composition can be incorporated with the brace as a removable insert or be fully integrated into the brace. In one embodiment, the brace includes a pouch configured to retain a nitroglycerin-containing composition, preferably in the form of a transdermal patch, against the skin of the subject wearing the brace. In such an embodiment, the nitroglycerin-containing composition can be readily removed from the pouch and replaced. The pouch can be of any configuration so long as it provides a secure location for the nitroglycerin-containing composition and maintains the compositions against the skin of the subject wearing the brace. Such a pouch can be partially open so that the nitroglycerin patch can contact the skin freely, or can include a permeable membrane or mesh that allows the drug to adequately contact the skin surface.

Generally, when the cost of the orthopedic support materials is high, it is preferable that the nitroglycerin-containing composition be replaceable. However, regardless of the cost of the orthopedic support materials, the nitroglycerin-containing composition can be integrated into the orthopedic support in such a way that it cannot be readily removed and replaced. In one embodiment, the nitroglycerin-containing composition can be sewn into the material of the orthopedic support. In another embodiment the orthopedic support can act as a backing layer onto which the nitroglycerin-containing composition is disposed and adhered.

Another type of brace which can be used in the present invention is an elastic or flexible sleeve. The sleeves can be made of any materials known in the art so long as they provide sufficient elasticity and flexibility for the desired application, e.g. application to a joint. The nitroglycerin-containing composition can be sewn into the sleeve or can be integrated directly into the sleeve, with the sleeve material acting as a backing layer for the nitroglycerin-containing composition

In another embodiment, the orthopedic support structure is an athletic support tape. When wrapped around the damaged musculoskeletal tissue, the tape can provide orthopedic support. The nitroglycerin-containing composition can be incorporated or integrated into the adhesive portion of the tape, or a portion of the tape. The tape can be manufactured by depositing the nitroglycerin-containing adhesive composition onto the tape substrate and allowing the nitroglycerin-containing composition to dry. It is a similar process to that described in Examples 1-7. When tape is used as the orthopedic support structure, the tape substrate can act as a backing layer.

As mentioned, the present invention can be used to provide orthopedic support and delivery of nitroglycerin for both short and long periods of time. For reduced pain and improved function, the nitroglycerin-containing composition can be formulated to provide sustained delivery of nitroglycerin over a continuous period of time of from 4 hours to 7 days. In another embodiment, the nitroglycerin-containing composition is formulated to deliver the nitroglycerin for a continuous period of from about 1 to about 3 days. In a further embodiment, the nitroglycerin-containing composition is formulated to deliver the nitroglycerin for a continuous period of from about 12 hours to 24 hours. The systems of the present invention can be continuously used over an administration period of from about 1 week to about 1 year. In one embodiment, a continuous administration period can last from about 1 day to about 24 weeks. As stated above, for the purposes of the present invention, planned regular drug holidays can be incorporated into an administration period without destroying its continuous nature.

### EXAMPLES

### Examples 1-7

Nitroglycerin-Containing Compositions

Several prototype nitroglycerin-containing compositions are prepared in accordance with embodiments of the present invention with the starting components listed in Table 1.

TABLE 1

| | Nitroglycerin | Ethyl Acetate | DuroTak 87-2194 (liquid) |
|---|---|---|---|
| Example | (Wt %) | (Wt %) | (Wt %) |
| 1 | 2.6% | 7.7% | 89.8% |
| 2 | 3.4% | 10.1% | 86.6% |
| 3 | 3.8% | 11.4% | 84.8% |
| 4 | 4.2% | 12.4% | 83.4% |
| 5 | 4.9% | 14.7% | 80.4% |
| 6 | 5.9% | 17.6% | 76.5% |
| 7 | 6.3% | 18.6% | 75.1% |

The nitroglycerin-containing compositions of Examples 1-6 are prepared in the following manner:

1. Nitroglycerin is diluted in the DuroTak 87-2194 adhesive and ethyl acetate solvent forming a nitroglycerin-containing composition. (see Table 1 for examples of dilution ratios)

2. The nitroglycerin-containing composition of the adhesive blend is formed onto a release liner using a mechanical coater.

3. The coated release liner is then passed through an oven which causes the solvent (e.g. ethyl acetate and the solvent present in the liquid DuroTak) to evaporate, forming a solid, tacky layer of adhesive matrix that contains nitroglycerin dispersed in an adhesive matrix.

4. A polyethylene film is then laminated to the adhesive matrix to act as a backing layer.

The above described manufacturing process can be varied such that the nitroglycerin-containing composition is laminated to or coated onto a portion of the orthopedic support structure. In such an embodiment the orthopedic support structure would act as a backing layer, providing support for the nitroglycerin-containing composition.

### Examples 8

Device to Treat a Stress Fracture Around a Joint

An athlete experiencing pain in the ankle which occurs during normal walking maneuvers is diagnosed with a stress fracture. Specifically, on examination, pain is reproduced upon squeezing the back of the heel on both sides. X-ray of the left ankle reveals a hairline calcaneal stress fracture. A nitroglycerin-releasing supportive sleeve applied which fits around the ankle and releases nitroglycerin in a controlled fashion over time, which enables the fracture to repair more quickly and reduces the pain associated with the fracture. This results in decreased pain and a shorter recovery time.

### Examples 9

Device to Treat a Long Bone Fracture

A sports related injury resulting in a lower leg deformity is confirmed by X-ray to be a simple fracture of the mid-shaft of the right tibia and fibula. The 0patient undergoes open reduction surgery, and the fracture is stabilized using a plate and pins. In the operating theater, prior to application of a temporary plaster cast, a nitroglycerin-containing composition integrated with a bandage is applied to the fracture site which allows controlled release of nitroglycerin over several days. After five days, the plaster cast is revised with a fiberglass cast, and the bandage and nitroglycerin-containing composition is also renewed at this time.

### Examples 10

Device to Treat a Tendon

A subject complaining of pain in the wrist demonstrates tenderness near the anatomical snuffbox, and shows a positive Finklestein' test. A diagnosis of de Quervain's tendonintis is made, and the subject is prescribed a brace with pouch for providing transdermal delivery of nitroglycerin via a nitroglycerin-containing composition. After a period of about 24 hours, nitroglycerin-containing composition in the pouch is replaced with new nitroglycerin-containing composition. After three weeks, the subject is asymptomatic.

### Examples 11

Device to Treat a Muscle

A subject experiencing stabbing pain to the left posterior thigh experiencing considerable pain upon walking, and demonstrating significant tenderness along the biceps femoris muscle with associated muscle spasm and mild swelling is diagnosed with a Grade 2 hamstring tear. The subject is immediately treated using ice packs and a compression bandage with an integrated nitroglycerin-containing composition. After four weeks of gentle physical therapy in combination with the compression bandage and nitroglycerin therapy, the patient resumed normal sporting activities.

### Examples 12

Device to Support a Joint

A subject experiencing pain in the right shoulder demonstrates mild tenderness over the shoulder tip, and an MRI shows a small tear in the supraspinatus tendon. A supportive sports tape is applied to the shoulder. The tape includes a nitroglycerin-containing composition layer which allows controlled release of nitroglycerin over time. This tape serves to physically prevent excess or abnormal movement, and the nitroglycerin assists in the reducing pain and tenderness of the affected tendon.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A system for relieving pain associated with damaged musculoskeletal tissue, comprising:
an orthopedic support structure which provides mechanical support to the damaged musculoskeletal tissue, and
a nitroglycerin-containing composition capable of delivering nitroglycerin through a skin surface to the damaged musculoskeletal tissue,
wherein the orthopedic support structure maintains the nitroglycerin-containing composition in contact with the skin.

2. The system of claim 1, wherein the orthopedic support structure includes an adhesive component for maintaining the nitroglycerin composition in contact with the skin.

3. The system of claim 1, wherein the orthopedic support structure includes a pouch capable of holding the nitroglycerin-containing composition, said pouch being configured to maintain the nitroglycerin composition in contact with the skin.

4. The system of claim 1, wherein the orthopedic support structure includes a recessed area which is capable of containing the nitroglycerin composition so as to maintain the nitroglycerin composition in contact with the skin.

5. The system of claim 1, wherein the orthopedic support structure is configured to support a human joint.

6. The system of claim 5, wherein the human joint is a knee, an elbow, an ankle, a wrist, or a shoulder.

7. The system of claim 1, wherein the nitroglycerin-containing composition is configured as a transdermal patch.

8. The system of claim 7, wherein the transdermal patch has a drug delivery zone defined by the area where the nitroglycerin-containing composition contacts the skin surface, said drug delivery zone having an area of at least 2.5 cm².

9. The system of claim 8, wherein the drug delivery zone has an area of from 2.5 cm² to 100 cm².

10. The system of claim 7, wherein the transdermal patch is an adhesive matrix patch.

11. The system of claim 7, wherein the transdermal patch is formulated to deliver the nitroglycerin from the nitroglycerin-containing composition for a period of from 4 hours to 7 days.

12. The system of claim 11, wherein the period is from 1 day to 3 days.

13. The system of claim 11, wherein the period is from 12 hours to 24 hours.

14. The system of claim 7, wherein the transdermal patch is formulated to deliver from 10 μg/hour to about 60 μg/hour.

15. The system of claim 1, wherein the damaged musculoskeletal tissue is tendon.

16. The system of claim 1, wherein the tendon is damaged as a result of a tendinopathy.

17. The system of claim 1, wherein the damaged musculoskeletal tissue is muscle.

18. The system of claim 1, wherein the damaged musculoskeletal tissue is bone.

19. The system of claim 1, wherein the damaged musculoskeletal tissue is ligament.

20. The system of claim 1, wherein the nitroglycerin-containing composition is formulated to deliver nitroglycerin at from about 5 μg/hour to about 85 μg/hour.

21. A method for treating damaged musculoskeletal tissue, comprising:
fitting an orthopedic support structure to a subject which mechanically supports to the damaged musculoskeletal tissue, and
applying a nitroglycerin-containing composition to a skin site proximate the damaged musculoskeletal tissue, said composition capable of delivering nitroglycerin through a skin surface to the damaged musculoskeletal tissue, wherein the orthopedic support structure is configured to maintain the nitroglycerin-containing composition in contact with the skin.

22. The method of claim 21, wherein the orthopedic support structure includes an adhesive component for maintaining the nitroglycerin composition in contact with the skin.

23. The method of claim 21, wherein the orthopedic support structure includes a pouch capable of holding the nitroglycerin-containing composition, said pouch being configured to maintain the nitroglycerin composition in contact with the skin.

24. The method of claim 21, wherein the orthopedic support structure includes a recessed area which is capable of containing the nitroglycerin composition so as to maintain the nitroglycerin composition in contact with the skin.

25. The method of claim 21, wherein the orthopedic support structure is configured to support a human joint.

26. The method of claim 25, wherein the human joint is a knee, an elbow, an ankle, a wrist, or a shoulder.

27. The method of claim 21, wherein the nitroglycerin-containing composition is configured as a transdermal patch.

28. The method of claim 27, wherein the transdermal patch has a drug delivery zone defined by the area where the nitroglycerin-containing composition contacts the skin surface, said drug delivery zone having an area of at least 2.5 cm².

29. The method of claim 28, wherein the drug delivery zone has an area of from 2.5 cm² to 100 cm².

30. The method of claim 27, wherein the transdermal patch is an adhesive matrix patch.

31. The method of claim 27, wherein the transdermal patch is formulated to deliver the nitroglycerin from the nitroglycerin-containing composition for a period of from 4 hours to 7 days.

32. The method of claim 31, wherein the period is from 1 day to 3 days.

33. The method of claim 31, wherein the period is from 12 hours to 24 hours.

34. The method of claim 27, wherein the transdermal patch is formulated to deliver from 10 μg/hour to about 60 μg/hour.

35. The method of claim 21, wherein the damaged musculoskeletal tissue is tendon.

36. The method of claim 21, wherein the tendon is damaged as a result of a tendinopathy.

37. The method of claim 21, wherein the damaged musculoskeletal tissue is muscle.

38. The method of claim 21, wherein the damaged musculoskeletal tissue is bone.

39. The method of claim 21, wherein the damaged musculoskeletal tissue is ligament.

40. The method of claim 21, wherein the nitroglycerin-containing composition is formulated to deliver nitroglycerin at from about 5 μg/hour to about 85 μg/hour.
